(19) United States
(12) Patent Application Publication
(10) Pub. No.: US 2020/0315235 A1
(43) Pub. Date: Oct. 8, 2020

(54) Normalization of the Intestinal Microbiota Composition in Infants or Toddlers Fed with an Amino Acid-Based Nutritional Composition

(71) Applicant: N. V. Nutricia, Zoetermeer (NL)

(72) Inventors: Marleen Theodora Johanna Van Ampting, Utrecht (NL); Harm Johannes Wopereis, Amersfoort (NL)

(21) Appl. No.: 16/339,874

(22) PCT Filed: Oct. 5, 2016
(86) PCT No.: PCT/NL2016/050690

§ 371 Date: Apr. 5, 2019

(51) Int. Cl. A23L 33/00; A23L 33/21; A23L 33/135; A23L 33/12; A23L 33/175; A61K 35/745; A61K 9/00

(52) U.S. Cl. CPC A23L 33/40; A23L 33/21; A23L 33/135; A23L 33/12; A23Y 2300/29; A61K 35/745; A61K 9/0095; A23V 2002/00; A23L 33/175

(57) ABSTRACT

The invention pertains to a nutritional composition comprising indigestible fiber and a lactic acid producing bacterium for normalizing the intestinal microbiota in amino acid-based formula fed infants or toddlers.

### BACKGROUND OF THE INVENTION

Intestinal microbiota composition (microbial gut flora) is recognized as being of primary importance for the health and wellbeing of individuals. There have been many research efforts directed to the defense mechanisms of the gastro-intestinal system, which can be seen as a biotope of microorganisms living in symbiosis with each other, as well as with the human body.

It is well known that the food consumed by an individual has a high impact on the intestinal microbiota. In addition, parameters of life quality and location, such as the presence of water with (or without) high hygiene standards, or living in urban or rural environments, also have a distinct influence on the intestinal microbiota of an individual.

The gut microbiota of infants establishes itself during birth and in the first period of life. It is widely accepted that breast fed infants have a relatively high number of lactic acid producing bacteria in their intestines, among which bifido bacteria. These bacteria are believed to help in further maturation of the intestines, but also in stimulating and maturation of the immune system.

Many new and exciting relations between body functions and the intestinal microbiota have been identified recently, which range from effects of the intestinal microbiota on obesity, on neurological effects, as well as others. It is therefore important for formula fed infants to maintain a healthy intestinal microbiota, which resembles as much as possible the intestinal microbiota of breast fed infants.

Infants fed with a bovine milk protein based formula are known to have a reduced quantity of lactic acid producing bacteria in their intestines, relative to the other bacteria, compared to breast-fed infants. This leads e.g. to an increase in fecal pH. In the prior art, dietary fibers have been added to bovine milk protein based formula. These dietary fibers, among which galactooligosaccharides and fructooligosaccharides, have the effect of stimulating the intestinal growth of lactic acid producing bacteria.

A considerable group of infants and toddlers must rely on amino acid-based nutritional compositions. For example, food allergic infants, in particular non-IgE mediated cows milk allergic infants, are dependent on amino acid-based formula. Also, infants and toddlers with hereditary diseases affecting amino acid metabolism depend on specific amino acid-based nutritional compositions, which may be depleted for the amino acid(s) they cannot metabolize.

The inventors now discovered that the intestinal microbiota of amino acid-based formula fed infants is different from breast fed infants as well as from normal cows milk-based formula fed infants. This makes the group of amino acid-based formula fed infants a sub-group of infants different to cows milk protein based formula fed infants. As the identified difference between amino acid-based formula fed infants and breast fed infants is even larger than the difference between breast-fed infants and cows milk-based formula fed infants, it is important to establish a healthy, normal intestinal microbiota also in these patient groups

Since not all dietary fibers stimulate the growth of lactic acid producing bacteria and not all lactic acid producing bacteria are equally capable of fermenting dietary fibers the present inventors hypothesized that adding suitable lactic acid producing bacteria to fermentable dietary fibers may stimulate the normalization of the intestinal microbiota in amino acid-based nutrition fed patients.

### SUMMARY OF THE INVENTION

The present inventors surprisingly discovered that the intestinal microbial composition (intestinal flora or intestinal microbiota) in amino acid-based formula fed infants and toddlers is different from the intestinal microbiota of cows milk-based infant formula fed infants, as well as from breast-fed infants. It was hitherto unknown, and could not have been predicted, whether amino-acid based formula fed infants would have a different intestinal microbiota than protein-fed infants, and if so, how the intestinal microbiota would be different. Surprisingly, the inventors discovered that a combination of indigestible fiber and a lactic acid producing bacterium normalizes the intestinal microbiota of this specific patient group.

The invention thus pertains to a nutritional composition comprising indigestible fiber and a lactic acid producing bacterium for use in normalizing the intestinal microbiota in amino acid-based formula fed infants or toddlers.

### DESCRIPTION OF FIGURES

FIG. 1: Fecal count of Bifidobacteria in at start of the intervention (t=0) and after 8 weeks of intervention. The reference values of breast milk fed infants is depicted in the grey arced area.

FIG. 2. Fecal counts of Clostridiumcluster XIVa at start of the intervention (t=0) and after 8 weeks of intervention. The reference values of breast milk fed infants is depicted in the grey arced area.

### DETAILED DESCRIPTION OF THE INVENTION

The present inventors discovered that infants who are using amino acid-based formula as a main source of protein often suffer from a dysbiosis in the intestinal microbiota. The balance of typical breast-fed infant fecal flora is high in Bifidobacteria and low in adult like strains such as members of Clostridium cluster XIV.

Terms like amino acid-based formula, amino acid-based diet and the like describe in the present context a nutritional composition wherein the protein source comprises at least 70 wt. % free amino acids, preferably at least 80 wt. %, even more preferably at least 90 wt. % free amino acids and most preferably between 90 and 100 wt. % free amino acids. Examples of amino acid-based nutritional compositions are Neocate and Anamix Infant (both Nutricia Advance Medical Nutrition).

The present composition is specifically intended for use in infants and/or toddlers which are fed amino acid-based compositions. Infants have an age of 0-12 months, toddlers have an age of 13-36 months. In one embodiment according to the present invention, the subject is an infant or toddler of 0-36 months. Preferably, the subject is an infant of 0-12 months. In the below, where “infant” is written, toddlers are intended equally, unless otherwise mentioned.

Normal, breast fed or cows milk fed children obtain most of their amino acids from protein, which is hydrolyzed in the digestive tract to yield free amino acids. As such, most of the amino acids available to the infant is consumed in the form of protein.

Amino acid-based formula fed infants are infants who consume on a daily basis in their diet at least 50 wt. % of the amino acid intake in the form of free amino acids, preferably at least 60, 70, 80, 90 wt. % of the amino acid intake is in the form of free amino acids, most preferably about 100 wt. %. Further preferably, between 80-100 wt. % of the daily amino acid intake is in the form of free amino acids. Amino-acid-based formula fed infants generally have a reason to avoid consumption of protein. Such reasons may include allergies to specific protein, or metabolic diseases which make that certain amino acids present in protein cause adverse physical reactions. Infants who generally rely on amino acid-based formula are in particular infants who have a food allergy, or who have a disorder of the amino acid metabolism.

Infants suffering from allergy may have any allergy, but in particular, infants allergic to cows milk (cows milk allergy; CMA) rely on amino acid-based infant formula, and are infants whose intestinal microbiota particularly benefits from the use of the present composition.

Infants who have a disorder of the amino acid metabolism also generally rely on amino acid-based formula, because protein generally comprises also the specific amino acid which causes adverse reactions. Such disorders comprise among others phenylketonuria, maple syrup urine disease, glutaric academia, and methylmalonic academia.

The inventors further found (as shown in example 1) that the intestinal microbiota of amino acid-based formula fed infants is different from formula fed infants fed with milk protein-based formula. This difference was large and led the inventors to the hypothesis that the intestinal microbiota was influenced by the amino acid-based formula. Amino acid-based formula fed infants had a (much) lower proportion of lactic acid producing bacteria, and a higher proportion of bacterial members of Clostridium cluster XIV, than breast-fed infants.

Since the intestinal microbiota is deemed very important for a diverse set of health issues among which intestinal maturation, development of the immune system, gut permeability, and others, the inventors have developed a composition that is capable of normalizing the intestinal microbiota.

Normalizing the intestinal microbiota in this context means that the composition of the intestinal microbiota is altered so as to more closely resemble the intestinal microbiota of breast fed infants. That is, the dysbiosis present in amino acid-based formula fed infants is alleviated. Or in other words ‘normalizing the intestinal microbiota’ means the treatment of dysbiosis in amino acid-based formula fed infants. Thus, the present composition increases the proportion of lactic acid producing bacteria, and decreases the proportion of clostridium bacteria, in the intestinal microbiota of amino acid-based formula fed infants. Breast-fed infants are understood to mean infants that are fed human milk, preferably from their natural mother.

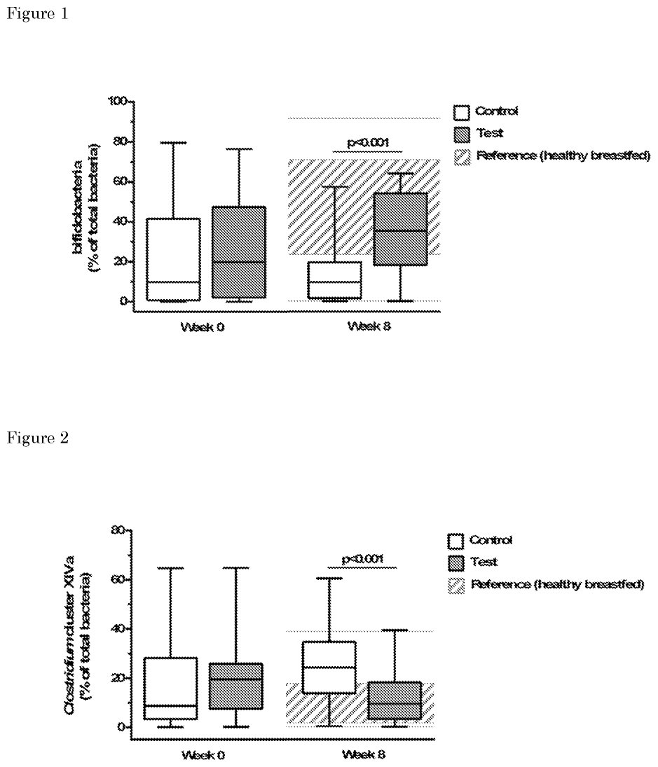

Surprisingly, as shown in example 2, the inventors found that the simultaneous administration of an indigestible fiber and a lactic acid producing bacterium resulted in treatment of the dysbiosis in infants fed with an amino acid-based formula. FIG. 1 shows the percentage of Bifidobacteria before (t=0) and after 8 weeks of substitution of the regular diet with a composition according to the invention, in comparison to a control (amino acid-based formula without indigestible fiber and without lactic acid producing bacterium). After 8 weeks of supplementation, the test group was within the range of Bifidobacteria counts in normal (breast fed, age-matched) infants while the control group still displayed strong dysbiosis. The difference between test group and control group was statistically significant.

Similar surprising results were found with the bacterial members of Clostridium cluster XIV that are supposed to have a negative effect on intestinal health of infants. Bacterial members of Clostridium cluster XIV are normally found in more adult intestinal flora, and present only in a minor proportion in infants. However, upon substitution of the regular diet with a composition according to the invention, the test group significantly decreased the clostridium cluster of bacteria compared to the control. As such, the microbiota was normalized compared to age-matched breast milk-fed infants, also with respect to clostridium bacteria.

### Indigestible Fiber

The present composition comprises indigestible fiber. Indigestible fiber in this context means indigestible oligo- and polysaccharides which are not or only partially digested by the endogenous human digestive system by the action of acids or digestive enzymes present in the human upper digestive tract (i.e. small intestine and stomach). Indigestible fiber can be broken down by the fermentive action of the intestinal microbiota. This fermentation results in the formation of one or more organic acids, preferably lactic acid, butyric acid, propionic acid and/or acetic acid.

Because the indigestible fiber serves as a food source for at least lactic acid producing bacteria present in the intestinal microbiota, growth of the intestinal lactic acid producing bacteria is preferentially stimulated. Lactic acid producing bacteria in this context include preferably microorganisms from the genus of Bifidobacterium and/or Lactobacillus, In preferred embodiments, growth of Bifidobacteria is stimulated, most preferably Bifidobacterium breve. In amino acid-based formula fed infants, a higher proportion of bifidobacteria and/or lactobacilli (expressed as % of total bacteria) stimulates the formation of a normal intestinal microbiota comparable to the intestinal microbiota of breast fed infants, and results in health benefits.

Indigestible fiber can be (water-) soluble or (water-) insoluble indigestible fiber. Preferably, indigestible fiber is (water-) soluble indigestible fiber. Soluble, in this context, refers to a solubility in water at 20° C. of at least 0.1 g/100 ml, preferably at least 0.5 g/100 ml.

Indigestible fiber in the present context is preferably at least one, more preferably at least two, types of indigestible fiber selected from the group consisting of fructo-oligosaccharides (including inulins), galacto-oligosaccharides (including transgalacto-oligosaccharides), xylo-oligosaccharides, arabino-oligosaccharides, arabinogalacto-oligosaccharides, gluco-oligosaccharides (including cyclodextrins, gentio- and nigero-oligosaccharides and indigestible polydextrose), chito-oligosaccharides, glucomanno-oligosaccharides, galactomanno-oligosaccharides (including partially hydrolyzed guar gum), mannan-oligosaccharides, fuco-oligosaccharides, sialic acid comprising oligosaccharides, uronic acid oligosaccharides, (including galcturonic acid oligosaccharides and pectin degradation products).

Preferably the present composition according to the invention comprises indigestible fiber which results in enhanced lactic acid production and/or increases the growth of lactic acid producing bacteria. The indigestible fiber is preferably selected from the group consisting of galactooligosaccharide, fructooligosaccharide, fructopolysaccharide, fucosyloligosaccharide, polydextrose, acacia gum or derivatives thereof, and pectin or derivatives thereof. Further preferably, the indigestible fiber is selected from the group consisting of the group consisting of fructooligosaccharides, non-milk derived fucosyloligosaccharides and polydextrose.

In the context of indigestible fiber as used herein, “oligosaccharide” includes short chain oligosaccharides with a DP of at most 10, as well as long chain oligosaccharides, with a DP of more than 10, preferably more than 25, more preferably more than 50. Long chain oligosaccharides with a DP of more than 10 may also be called polysaccharides.

In preferred embodiments, the composition comprises at least two different types of indigestible fiber, preferably a combination of a short-chain oligosaccharide and a long-chain oligosaccharide. Further preferably, the weight ratio in a mixture of a short-chain oligosaccharide and a long-chain oligosaccharide, is between 25 and 0.05, more preferably between 20 and 1.

Acacia gum (GUM) or gum arabic is a soluble dietary fibre obtained from the stems and branches of Acacia senegal and A. seyal. It is composed mainly of complex polysaccharides (95%) that consist of highly branched galactan polymers, with galactose and/or arabinose side chains, possibly terminated by rhamnose or glucuronic acid residues.

More preferably the present composition comprises fructo-oligosaccharides, galacto-oligosaccharides and/or galacturonic acid oligosaccharides. In a preferred embodiment the composition comprises the mixture of fructo-oligosaccharides that was also used in example 2.

In another preferred embodiment the composition comprises a mixture short chain fructo-oligosaccharides (“scFOS”, with an average DP of at most 10) and long chain fructo oliogosaccharides (“lcFOS”, with an average DP of more than 50), or inulin, which is a mixture of scFOS and lcFOS. Preferably the ratio scFOS:lcFOS is between 20 and 1, more preferably 9:1.

Fructo-oligosaccharides are not derived from milk (like galactooligosaccharides) and are therefore highly suitable for use in amino acid based formula intended for cows milk allergic infants.

Preferably, the composition comprises 80 mg to 3 g indigestible fiber per 100 ml, more preferably 150 mg to 2 g, even more preferably 300 mg to 1.5 g indigestible fiber per 100 ml.

Based on dry weight, the composition preferably comprises 0.25 wt. % to 7.5 wt. % indigestible fiber (2.5-75 mg per gram dry weight), more preferably 0.5 wt. % to 5.5 wt. %, more preferably 1.0-4.0 wt. %, even more preferably 1.5 wt. % to 3.0 wt. %. A lower quantity of indigestible fiber is less effective in stimulating the lactic acid producing bacteria in the intestinal microbiota, whereas a too high quantity results in side-effects, such as bloating and abdominal discomfort.

### Lactic Acid Producing Bacteria

Immune effects of bacteria are highly species and/or strain specific. Therefore the inventors have performed extensive preclinical work in the field of identifying the most effective and safe species to be used as probiotic bacteria in the diet of amino acid based nutritional compositions.

Lactic acid producing bacteria in the present context preferably include bacteria from the genus of Bifidobacterium and/or Lactobacillus, preferably Bifidobacterium, such as preferably selected form the group consisting of Bifidobacterium breve, Bifidobacterium longum, Bifidobacterium infantis, and Bifidobacterium lactis. Most preferably, the lactic acid producing bacterium is Bifidobacterium breve.

Bifidobacterium breve (B. breve) is a Gram-positive, anaerobic, branched rod-shaped bacterium. B. breve preferably has at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849). Preferred B. breve strains are those isolated from the feces of healthy breast milk-fed infants.

Typically, lactic acid producing bacteria are commercially available from producers of lactic acid bacteria, but they can also be directly isolated from feces, identified, characterised and produced.

According to a preferred embodiment, the present composition contains at least one B. breve selected from the group consisting of B. breve Bb-03 (Rhodia/Danisco), B. breve M-16V (Morinaga), B. breve R0070 (Institute Rosell, Lallemand), B. breve BRO3 (Probiotical), B. breve BR92 (Cell Biotech), B. breve NCC2950 (CNCM 1-3865, DSM 20091, LMG 11613, YIT4065, FERM BP-6223 and CNCM 1-2219. Most preferably, the B. breve is selected from the group consisting of B. breve M-16V and B. breve CNCM 1-2219, most preferably B. breve M-16V. B. breve M-16V was deposited with the Belgian Co-ordinated Collections of Microorganisms (BCCM) and designated LMG 23729.

Preferably, the present composition comprises 10⁴ to 10¹², more preferably from 10⁵ to 10¹¹, most preferably from 10⁷ to 5×10¹⁰ colony forming units (cfu) of lactic acid producing bacteria per g of the total of indigestible fiber. In alternative preferred embodiments, the concentration of lactic acid producing bacterium is between 2.0×10⁸ and 2.0×10¹¹ cfu lactic acid producing bacterium per gram indigestible fiber.

The present composition preferably contains 10² to 10¹³ colony forming units (cfu) of lactic acid producing bacteria per gram dry weight of the present composition, preferably 10⁴ to 10¹², more preferably 10⁵ to 10¹¹, more preferably 10⁶ to 10¹¹, most preferably from 10⁷ to 10¹⁰ cfu lactic acid producing bacteria per gram dry weight of the present composition. The dose of lactic acid producing bacteria according to the present invention is preferably a daily dose of 10² to 10¹³, more preferably from 10⁵ to 10¹², most preferably from 10⁸ to 5×10¹⁰ colony forming units (cfu).

### Lipid Source

The present nutritional composition preferably comprises a lipid source. Lipid, in this context, is any mixture comprising at least 85 wt. %, preferably at least 90 wt. % triglycerides. As such, lipid should be considered to include both oil and fat. Fat is solid at room temperature, whereas oil is liquid or viscous.

The lipid source may comprise a plant-derived lipid (such as plant oils, among which olive, sunflower or rapeseed oil or coconut fat), an animal-derived lipid (such as milk fat, lard or fish oil), or a microbial lipid, such as obtained by fermentation of specific microorganisms (such as microbial ARA or DHA comprising oils).

The quantity of lipid source in the composition of the invention is 0-50 wt. %, based on dry weight, preferably 2-40 wt. %, more preferably 5-30 wt. %. Alternatively, the composition comprises 10-60 en. %, preferably 35-50 en. % lipid source.

In preferred embodiments, the lipid source comprises at least one long-chain polyunsaturated fatty acid (LC-PUFA). Preferably, LC-PUFA are polyunsaturated fatty acids with a chain length of 20 or 22 carbon atoms, most preferably eicosapentaenoic acid (EPA), docosahexaenoic acid (DHA) and/or arachidonic acid (AA). LC-PUFA are preferably present as a triglyceride mixture. A triglyceride is considered an LC-PUFA triglyceride if it comprises at least 1, preferably 2 or 3, and most preferably 3 LC-PUFAs.

Preferably, the lipid source comprises at least 0.1 wt. %, preferably at least 0.25 wt. %, more preferably at least 0.5 wt. %, even more preferably at least 0.75 wt. % LC-PUFA of the total glyceride-derived fatty acid content. The content of LC-PUFA triglyceride in the present composition, preferably does not exceed 15 wt. % of the total lipid content, more preferably does not exceed 10 wt. %, even more preferably does not exceed 5 wt. % of the total lipid content. LC-PUFA beneficially improves gut barrier function and is therefore believed to improve the efficacy of the indigestible fiber and lactic acid producing bacteria composition for normalizing the intestinal microbiota.

### Protein Source

A nutritional composition according to the invention preferably comprises a protein source. Preferably, said protein is non-allergenic to the target group of interest. Protein, in this context, refers to any material or mixture of materials comprising at least 90 wt. %, more preferably at least 95 wt. %, of amino acids. Protein may comprise native protein, denatured protein, hydrolyzed protein, peptides of at most 10 amino acids, as well as free amino acids. In preferred embodiments, protein comprises at least 70 wt. % free amino acids, more preferably at least 80 wt. %, more preferably at least 90 wt. %, even more preferably at least 95 wt,%, and most preferably about 100 wt. % free amino acids.

Free amino acids, in this context, refers to the standard amino acids known to exist in the human body, and forming among others active protein. Free amino acids may be present as in their neutral state, or in the form of a physiologically acceptable salt. Suitable salts are known in the art, and include for example sodium, potassium and chloride salts of amino acids.

In the composition according to the present invention, the content of the protein source is preferably between 2 and 40 wt. % protein, based on dry weight of the total composition, preferably between 11 and 18 wt. %, and even more preferably between 12 and 16 wt. % protein based on dry weight of the total composition. In alternative preferred embodiments, the protein content is 5-50 en. %, preferably 7.5-15 en. %.

### Carbohydrates

A composition according to the invention preferably comprises carbohydrates. Carbohydrates, in this context, are digestible carbohydrates, which comprise mono-, di- or tri-saccharides well-known in the art of nutrition, as well as digestible oligo- and polysaccharides, among which starch. In preferred embodiments, the carbohydrates include glucose, fructose and lactose.

In preferred embodiments, the composition comprises between 10 and 90 wt. %, based on dry weight, of carbohydrates, preferably between 50 and 70 wt. %. In alternative preferred embodiments, the composition comprises between 15 and 90 en. % carbohydrates, preferably between 40 and 55 en. %.

### Nutritional Compositions and Mode of Administration

The composition used in the present method is preferably administered enteral, more preferably orally. The composition of the invention can be a composition comprising indigestible fiber and a lactic acid producing bacterium, which composition can be portioned before use to be mixed in with any commercial infant formula, in which case feeding the mixture of infant formula and the composition of the invention results in normalization of the intestinal microbiota.

In a preferred embodiment the present composition is administered as, or is comprised in, a nutritional matrix, said nutritional matrix preferably containing a lipid, a protein and a carbohydrate component.

For infants or toddlers with the age between 0 and 36 months, preferably the lactic acid producing bacterium is embedded in a nutritional composition containing lipid source, carbohydrates and free amino acids as protein source.

In a preferred embodiment the present nutritional composition fulfills, or the present method comprises the administration of a nutritional composition which fulfills the requirements for feeding infants, particularly nutritional compositions containing between 10 and 60 en. % lipid source, between 5 and 50 en. % free amino acids as protein source, and/or between 15 and 90 en. % carbohydrate.

More preferably the nutritional composition comprises between 7.5 to 15 en. % protein source, 40 to 55 en. % carbohydrates, and 35 to 50 en. % lipid source. (en. % is short for energy percentage and represents the relative amount each constituent contributes to the total caloric value of the preparation).

The composition according to the invention can be a powder or a liquid. Preferably the composition is a powder composition to be reconstituted in water to form a drink or a thickened product with a viscous consistency or pudding like consistency. The advantage of a powder is that the shelf life of the composition with life bacteria will be longer then when the product would be a liquid.

Preferred embodiment is a powdered amino acid-based complete nutritional composition such as Neocate™ with at least 10⁸ CFU Bifidobacterium breve and at least 1 g fructooligosaccharides for use in the normalization of the intestinal microbiota in patients suffering from allergy.

The composition is preferably used as the main nutrition of the infant or toddler. That is, at least 75 wt. % of the total protein intake of the infant or toddler stems from the composition. Nourishment with the composition is preferably daily, more preferably 1-10 times a day, most preferably 3-8 time a day.

Neocate contains about 46 en. % lipids, 43 en. % carbohydrates and 11 en. % protein equivalent (free amino acids L-arginine L-aspartaat, L-leucine, L-lysineacetaat, L-glutamine, L-proline, L-valine, glycine, L-isoleucine, L-threonine, L-phenylalanine, L-tyrosine, L-serine, L-histidine, L-alanine, L-cystine, L-tryptofaan, L-methionine) and all vitamins an minerals necessary for optimal growth. In a preferred embodiment the composition further comprises per 100 g dry weight about 82 mg docosahexaenoic acid (DHA) and 82 mg arachidonic acid (AA).

The invention furthermore discloses a method for normalizing the intestinal microbiota in amino acid-based formula fed infants or toddlers, comprising daily feeding said infant or toddler with a composition comprising indigestible fiber and a lactic acid producing bacterium as defined above. In preferred embodiments, the method comprises feeding the infant or toddler with essentially only said composition as protein source.

### EXAMPLES

### Example 1 Average Bifidobacteria and Clostridium Cluster XIV Counts in the Feces of Infants Fed with Breast Milk, Bovine Milk-Based Formula and Amino Acid-Based Formula

The measurements were performed according to the methods described in example 2

The Bifido species are measured using the FISH-probe Bif164, see (http://probebase.csb.univie.ac.at/pb_report/probe/37)

ER/CC refers to the FISH—probe Erec482 (see: http://probebase.csb.univie.ac.at/pb_report/probe/963) and it recognizes (most of the) “Eubacterium rectale—Clostridium coccoides group (Clostridium cluster XIVa and XIVb also known as the family of Lachnospiraceae)”

Since the age of the infant can influence the intestinal microbiota, the average age of the infants used for this comparison was kept in the same range. The data come from multi-center trials thereby also equalizing the (potential) effect of the place of birth and local hygiene levels. The differences found in the intestinal microbiota of these infants can only be attributed to the nutrition they have consumed.

Conclusion: The results clearly show that amino acid fed infants have a less normal intestinal microbiota compared to protein based formula fed infants.

Human milk avg. 8.5 months old infants
bifidobacteria 55%
ER/CC 6.5%

Formula fed avg. 8 months old infants
Bifidobacteria 30.1%
ER/CC 38.1%

free amino acid fed: avg 8 months old infants
bifidobacteria 14.7%
ER/CC cluster was 26.6%

free amino acid fed: avg 8.5 months old infants
Bifidobacteria 13%
ER/CC 35.53%

### Example 2 Normalization of Microbiota in Amino-Acid-Fed Infants Shown in a Placebo Controlled Double Blind Clinical Study

Bifidobacteria and Clostridium cluster XIVa were used as markers to explore the concept that amino acid feeding with a symbiotic blend comprising a lactic acid producing bacterium and indigestible fiber may modify intestinal microbial composition (intestinal flora or intestinal microbiota) in infants with CMA to be close to that observed in healthy breastfed infants at a comparable age. Studies of intestinal flora in early life suggest that maturation to adult-like profiles may extend beyond 5 years of age. Bifidobacteria are typically abundant in healthy breastfed infants and show stable and increased levels in early childhood compared with adults. Clostridium cluster XIVa was selected as marker for this study because it typically comprises one of the first adult-like clusters appearing in the infant intestinal microbiota.

Methods: A multicenter, double-blind, randomized controlled trial was conducted in subjects <13 months old with non-IgE-mediated CMA. Healthy breastfed infants (age matched to the week-8 age of infants with CMA) were selected as a ‘healthy reference’ group. Allergic subjects were randomly allocated to receive test or control product for 8 weeks. The test product was a hypoallergenic, nutritionally complete amino acid based formula containing a prebiotic blend of fructo-oligosaccharides and the probiotic strain Bifidobacterium breve M-16V. The control product was an identical amino acid based formula without the prebiotic blend and without the probiotic strain. Products were identically packaged to ensure blinding.

Baseline assessments (week 0) included clinician reported medical history of presenting complaints by 24 hr recall. Stool samples for analysis were collected by parents/guardians at week 0, if possible under the supervision of a healthcare professional, and then at home at week 8. Samples collected into 10 mL stool containers (Greiner Bio-One, Kremsmünster, Austria) were frozen at −80° C. in the clinic or at −20° C. in a home freezer before transfer to the clinic storage facility.

The primary objective was to assess the effect of test product on the fecal levels of bifidobacteria and Clostridium cluster XIVa, determined by fluorescence in situ hybridization (FISH) analysis using 16S rRNA-targeted oligonucleotide probes, as described in [Clin Exp Allergy 2010; 40(5):795-804].

Results: 35 subjects were randomly allocated to test product and 36 to control product; the healthy reference group included 51 infants. All randomized subjects were included in intent to treat analyses. Levels of bifidobacteria (higher) (FIG. 1) and Clostridium cluster XIVa (lower) (FIG. 2) were significantly different between test and control products at week 8 (P<0.001). Microbiota levels in the test group were close to levels in the healthy reference group. There were no differences in reported adverse events between test and control groups.

Conclusion: The combination of dietary fiber and a Bifidobacterium breve was capable of normalizing the intestinal microbial dysbiose in amino acid-based formula fed infants. The proportion of lactic acid producing bacteria increased, and the proportion of clostridium bacteria decreased.

### Example 3. Composition for Use According to the Invention

TABLE 1
Composition

| Unit | per 100 g dry weight | per 100 ml¹ |
|---|---|---|
| energy [kcal] | 483 | 67 |
| protein (see table 2) [g] | 13 | 1.8 |
| % of total energy [en. %] | 10.8 | 10.8 |
| digestible carbohydrate [g] | 52.5 | 7.3 |
| sugars [g] | 4.7 | 0.65 |
| % of total energy [en. %] | 43.5 | 43.5 |
| lipid [g] | 24.5 | 3.4 |
| % of total energy [en. %] | 45.7 | 45.7 |
| saturated fat [g] | 8.9 | 1.2 |
| monounsaturated fat [g] | 9.6 | 1.3 |
| Polyunsaturated fat [g] | 4.8 | 0.67 |
| DHA [mg] | 110 | 15 |
| ARA [mg] | 110 | 15 |
| fibre²: scFOS + lcFOS [g] | 4.9 | 0.68 |
| Lactic acid bacteria³ | B. breve M-16V | B. breve M-16V |

¹14.7 g powder is dissolved in 100 ml water
²Ratio scFOS:lcFOS = 9:1
³B. breve M-16V: 1.9 × 10⁹ CFU per gram fibre

TABLE 2
Composition of protein source
The protein source was a mixture of free amino acids with
the following composition [g/100 g composition]:

| | |
|---|---|
| L-Alanine [g] | 0.6 |
| L-Arginine [g] | 1.0 |
| L-Aspartic acid [g] | 1.0 |
| L-Cystine [g] | 0.4 |
| L-Glutamine [g] | 1.3 |
| Glycine [g] | 0.9 |
| L-Histidine [g] | 0.6 |
| L-Isoleucine [g] | 0.9 |
| L-Leucine [g] | 1.6 |
| L-Lysine [g] | 1.1 |
| L-Methionine [g] | 0.2 |
| L-Phenylalanine [g] | 0.7 |
| L-Proline [g] | 1.1 |
| L-Serine [g] | 0.7 |
| L-Threonine [g] | 0.8 |
| L-Tryptophan [g] | 0.3 |
| L-Tyrosine [g] | 0.7 |
| L-Valine [g] | 1.0 |
| L-Carnitine [g] | 0.01 |

1. A nutritional composition comprising indigestible fiber and a lactic acid producing bacterium, wherein the nutritional composition is used for normalizing the intestinal microbiota in an amino acid-based formula fed infant or toddler.

2. The nutritional composition according to claim 1, wherein the lactic acid producing bacterium is a Bifidobacterium.

3. The nutritional composition according to claim 2, wherein the Bifidobacterium is selected from the group consisting of Bifidobacterium breve, Bifidobacterium longum, Bifidobacterium infantis, and Bifidobacterium lactis.

4. The nutritional composition according to claim 2, wherein the Bifidobacterium is Bifidobacterium breve M-16V.

5. The nutritional composition according to claim 1, wherein the concentration of lactic acid producing bacterium is between 2.0×10⁸ and 2.0×10¹¹ CFU lactic acid producing bacterium per gram indigestible fiber.

6. The nutritional composition according to claim 1, wherein the concentration of indigestible fiber is from 0.25-7.5 wt. %, based on the dry weight of the composition.

7. The nutritional composition according to claim 1, wherein the indigestible fiber is selected from the group consisting of galactooligosaccharide, fructooligosaccharide, fructopolysaccharide, fucosyloligosaccharide, polydextrose, acacia gum or derivatives thereof, and pectin or derivatives thereof.

8. The nutritional composition according to claim 1, wherein the indigestible fiber is selected from the group consisting of fructooligosaccharides, non-milk derived fucosyloligosaccharides and polydextrose.

9.-10. (canceled)

11. The nutritional composition according to claim 1, wherein the nutritional composition further comprises a lipid source.

12. The nutritional composition according to claim 11, wherein the lipid source comprises at least one long-chain polyunsaturated fatty acid (lcPUFA), preferably eicosapentaenoic acid (EPA), docosahexaenoic acid (DHA) and/or arachidonic acid (AA).

13. The nutritional composition according to claim 1, wherein the infant or toddler has a food allergy or a disorder of the amino acid metabolism.

14. The nutritional composition according to claim 13, wherein the food allergy is cow milk allergy (CMA).

15. The nutritional composition according to claim 13, wherein the disorder of the amino acid metabolism is phenylketonuria, maple syrup urine disease, glutaric academia, and methylmalonic academia.

16. A method for normalizing the intestinal microbiota in an amino acid-based formula fed infant or toddler, comprising administering an effective amount of the nutritional composition of claim 1 to the infant or toddler in need thereof.

17. The method according to claim 16, wherein the infant or toddler has a food allergy or a disorder of the amino acid metabolism.

18. The method according to claim 17, wherein the food allergy is cow milk allergy (CMA).

19. The method according to claim 17, wherein the disorder of the amino acid metabolism is phenylketonuria, maple syrup urine disease, glutaric academia, and methylmalonic academia.
